(19) United States
(12) Patent Application Publication
(10) Pub. No.: US 2024/0316238 A1
(43) Pub. Date: Sep. 26, 2024

(54) SYSTEM FOR DISINFECTING A CLOSEABLE SPACE

(71) Applicant: SIGNIFY HOLDING B.V., Eindhoven (NL)

(72) Inventor: TIES VAN BOMMEL, HORST (NL)

(21) Appl. No.: 18/270,049

(22) PCT Filed: Dec. 27, 2021
(86) PCT No.: PCT/EP2021/087678

§ 371 Date: Jun. 28, 2023

(30) Foreign Application Priority Data
Jan. 5, 2021 (EP) 21150193.7

(51) Int. Cl. A61L 9/20

(52) U.S. Cl. CPC A61L 9/20; A61L 2209/111; A61L 2209/12

(57) ABSTRACT

The invention provides a system (100) for disinfecting at least part of a closeable space (10), wherein the system (100) comprises one or more light sources (110), a sensor (120) and a control system (300), wherein in a first operational mode one or more of the one or more light sources (110) are configured to generate first light source light (111) having one or more wavelengths selected from the range of 100-380 nm, and wherein in a second operational mode one or more of the one or more light sources (110) are configured to generate second light source light (112) having a centroid wavelength selected from the range of 400-490 nm, wherein the first light source light (111) and the second light source light (112) is provided in the closeable space (10), wherein the sensor (120) is configured to detect a state or a change in state of the closeable space (10) and to provide a related sensor signal to the control system (300), wherein the control system (300) is configured to select the first operational mode during a first time period (21) when the closeable space (10) is configured in a closed state (11) and configured to select the second operational mode during a second time period (22) when the closeable space (10) is configured in an open state (13).

### FIELD OF THE INVENTION

The invention relates to a system for disinfecting at least part of a closeable space. The invention further relates to a structure comprising the system. The invention further relates to a kit of parts.

### BACKGROUND OF THE INVENTION

Disinfecting systems for closeable spaces may be known in the art. For instance, US20200206373A1 describes devices, methods, and systems for disinfection with light. In some examples, a lighting source is operable to provide light at wavelength range of about 380-420 nm. One or more sensors and a control system may be used to control operation of the lighting source, such as by adjusting the lighting source in response to various inputs.

### SUMMARY OF THE INVENTION

UV light has been used for disinfection for over 100 years. Wavelengths between about 190 nm and 300 nm may be strongly absorbed by nucleic acids, which may result in defects in an organism's genome. This may be desired for killing bacteria and viruses, but may also have undesired side effects for humans. Therefore the selection of wavelength of radiation, intensity of radiation and duration of irradiation may be limited in environments where people may reside such as offices, public transport, cinema's, restaurants, shops, etc., thus limiting the disinfection capacity. Especially in such environments, additional measures of disinfection may be advantageous to prevent the spread of bacteria and viruses such as influenza or novel (corona) viruses like COVID-19, SARS and MERS.

It is desired to protect yourself and others from the spread of bacteria, fungi and viruses such as influenza or against the outbreak of novel viruses like the recent COVID-19. Disinfection systems may facilitate such efforts by providing cleaner and safer environments.

For instance, disinfection systems may use one or more anti-microbial and/or anti-viral means to disinfect a space or an object. Examples of such means may be chemical agents and disinfecting light, such as disinfecting light in the UV range.

Prior art disinfection systems may, however, also raise concerns. For instance, the chemical agents and/or disinfecting light may also be harmful for people and pets. Further, people may doubt whether (sufficient) disinfection has actually occurred, which may lead to a feeling of unsafety.

Further, prior art disinfection systems may use continuous external indicator lighting to inform a user that a space has been disinfected. However, such indicator lighting may be considered annoying, especially when, for instance, multiple objects in a room have external indicator lighting, and may be excessively energy-consuming, especially when the space is only infrequently used.

Hence, it is an aspect of the invention to provide an alternative disinfection system, which especially further at least partly obviates one or more of above-described drawbacks. The present invention may have as object to overcome or ameliorate at least one of the disadvantages of the prior art, or to provide a useful alternative.

Hence, in a first aspect the invention may provide a system (or: disinfection system”) for disinfecting at least part of a closeable space (also: “space”). In embodiments, the system may comprise one or more of one or more light sources, optionally a sensor, and a control system (also: “controller”). In a first operational mode, one or more of the one or more light sources may be configured to generate first light source light, especially first light source light having one or more wavelengths selected from the range of 100-380 nm. In a second operational mode, one or more of the one or more light sources may be configured to generate second light source light having a centroid wavelength selected from the range of 400-490 nm. Especially, the first light source light and the second light source light may be provided in (and to) the closeable space, especially in the first operational mode and the second operational mode, respectively. In embodiments, the (optional) sensor may be configured to detect a state or a change in state of the closeable space and (be configured) to provide a related sensor signal to the control system. In particular, the state or the change of the state may be indicative of when the space is closed and when the space is opened e.g. indicative of when a door is closed and when a door is opened. Especially, the sensor may be configured to detect whether the closeable space is in a closed state or in an open state and to provide a related sensor signal to the control system. In embodiments, the control system may be configured to select the first operational mode during a first time period when the closeable space is configured in a closed state. In further embodiments, the control system may be configured to select the second operational mode during a second time period when the closeable space is configured in an open state, especially when the closeable space changes from the closed state to a (partially) open state, i.e., at t=0 when opening the closeable space. Hence, the control system may in embodiments be functionally coupled to the (optional) sensor.

In particular, the system of the invention may provide the benefit that the closeable space is not only disinfected using the first light source light, but that a user opening the closeable space will also be notified of the cleaning via the second light source light. Hence, in embodiments the invention may also be used to indicated that a closeable space has been disinfected. Further, in a safe way the closeable space may be disinfected. Yet further, in embodiments it may be possible that the closeable space may be disinfected only for a predetermined period.

Therefore, in embodiments the invention may provide a system comprising a colored light source and a disinfection source for providing disinfection. The disinfection source may be a UV light source for providing UV light. The colored light source may especially be a (short wavelength) blue light source providing (short wavelength) blue light. The system may further comprise in embodiments a sensor for detecting a state or a change in state of the closeable space, especially whether the closeable space is open or close. When the closeable space is closed then the UV (LED) light source may provide the UV light source light for disinfection of the space of any items in the space. When the space is opened, in embodiments the UV light may immediately be switched off, while the blue light may be switched on. During the opening of the drawer the blue light may in embodiments also be switched off. In this way, the UV light for disinfection may be used in a safe way (because of shielding), while the user may have the impression that the disinfection has been executed. In particular, UV light may often be associated with a blue and/or purple color. Hence, by (briefly) providing the blue and/or purple color when the closeable space is opened, the user may be conveyed the sense that the space has indeed been disinfected, while the (second light source) light the user is exposed to is safe.

In specific embodiments, the invention may provide a system for disinfecting at least part of a closeable space, wherein the system comprises one or more light sources, a sensor and a control system, wherein in a first operational mode one or more of the one or more light sources are configured to generate first light source light having one or more wavelengths selected from the range of 100-380 nm, and wherein in a second operational mode one or more of the one or more light sources are configured to generate second light source light having a centroid wavelength selected from the range of 400-490 nm, wherein the first light source light and the second light source light may be provided in the closeable space, wherein the sensor is configured to detect a state or a change in state of the closeable space and to provide a related sensor signal to the control system, wherein the control system is configured to select the first operational mode during a first time period when the closeable space is configured in the closed state and configured to select the second operational mode during a second time period when the closeable space is configured in the open state. Hence, the invention may provide a system for disinfecting at least part of a closeable space.

The term “closeable space” may refer to any space that may, during normal use, be closed for periods of time. For instance, the closeable space may comprise a room, the inside of a cabinet, the inside of a drawer, the inside of a car, the inside of a box, a containment, et cetera, especially a room, or especially a containment. The closeable space in the closed state may in embodiments have a volume of at least 0.25 dm³, even more especially at least 0.5 dm³, such as at least 1 dm³. In embodiments, the closeable space may have in the closed state at maximum about 15 m³, such as at maximum about 10 m³, like at maximum about 5 m³. In other embodiments, however, the closeable space may have in the closed state a volume of at least about 5 m³. In further specific embodiments, the closeable space may comprise a room, wherein the room has a volume of at least 10 m³, such as at least 20 m³. especially at least 25 m³. In further embodiments, the room may have a volume of at most 150 m³, such as at most 125 m³, especially at most 100 m³. In further embodiments, the closeable space may comprise a containment, wherein the containment has a volume of at least 1 dm³, such as at least 3 dm³. especially at least 5 dm³. In further embodiments, the containment may have a volume of at most 3 m³, such as at most 2 m³, especially at most 1 m³.

The system may comprise one or more light sources. The term “light source” may in principle refer to any light source known in the art. It may be a conventional (tungsten) light bulb, a low pressure mercury lamp, a high pressure mercury lamp, a fluorescent lamp, a LED (light emissive diode). In a specific embodiment, the light source may comprise a solid state LED light source (such as a LED or laser diode). The term “light source” may also relate to a plurality of light sources, such as 2-200 (solid state) LED light sources. Hence, the term LED may also refer to a plurality of LEDs. Further, the term “light source” may in embodiments also refer to a so-called chips-on-board (COB) light source. The term “COB” especially refers to LED chips in the form of a semiconductor chip that is neither encased nor connected but directly mounted onto a substrate, such as a PCB. Hence, a plurality of light semiconductor light sources may be configured on the same substrate. In embodiments, a COB is a multi LED chip configured together as a single lighting module.

The term “light source” may refer to a semiconductor light-emitting device, such as a light emitting diode (LEDs), a resonant cavity light emitting diode (RCLED), a vertical cavity laser diode (VCSELs), an edge emitting laser, etc. . . . . The term “light source” may also refer to an organic light-emitting diode, such as a passive-matrix (PMOLED) or an active-matrix (AMOLED). In a specific embodiment, the light source comprises a solid-state light source (such as a LED or laser diode). In an embodiment, the light source comprises a LED (light emitting diode). The terms “light source” or “solid state light source” may also refer to a superluminescent diode (SLED).

The term LED may also refer to a plurality of LEDs. Further, the term “light source” may in embodiments also refer to a so-called chips-on-board (COB) light source. The term “COB” especially refers to LED chips in the form of a semiconductor chip that is neither encased nor connected but directly mounted onto a substrate, such as a PCB. Hence, a plurality of semiconductor light sources may be configured on the same substrate. In embodiments, a COB is a multi LED chip configured together as a single lighting module.

The term “light source” may also relate to a plurality of (essentially identical (or different)) light sources, such as 2-2000 solid state light sources. In embodiments, the light source may comprise one or more micro-optical elements (array of micro lenses) downstream of a single solid-state light source, such as a LED, or downstream of a plurality of solid-state light sources (i.e. e.g. shared by multiple LEDs). In embodiments, the light source may comprise a LED with on-chip optics. In embodiments, the light source comprises a pixelated single LEDs (with or without optics) (offering in embodiments on-chip beam steering).

In embodiments, the light source may comprise any source of radiation that emits in the UV wavelength range. Especially, in embodiments the light source may comprise a mercury discharge lamp, such as a low pressure mercury discharge lamp, a medium pressure mercury discharge lamp or a high pressure mercury discharge lamp. Depending on the pressure of the mercury discharge lamp, the emitted radiation may have one or more emission peaks at 184 nm, 254 nm, 365 nm, 405 nm, 436 nm, 546 nm, 578 nm, and 650 nm. Alternatively, the light source may comprise an excimer (or exciplex) discharge based light source. In embodiments, the light source may comprise a KrBr excimer lamp which may provide radiation at 207 nm±15 nm. In alternative embodiments, the light source may comprise a KrCl excimer lamp which may provide radiation at 222 nm±15 nm. In alternative embodiments, the light source may comprise a Zn vapor discharge lamp which may provide radiation at 214 nm±15 nm. The wavelength ranges indicated above may be based on the full-width at half maximum (FWHM) of the emission bands. In other embodiments, the light source comprises a solid state light source, such as a UV LED.

The system, or apparatus, or device (see further also below) may execute an action in a “mode” or “operational mode” or “mode of operation”. Likewise, in a method an action, stage, or step may be executed in a “mode” or “operation mode” or “mode of operation”. This does not exclude that the system, or apparatus, or device may also be adapted for providing another operational mode, or a plurality of other operational modes. Likewise, this does not exclude that before executing the mode and/or after executing the mode one or more other modes may be executed. However, in embodiments a control system (see further also below) may be available, that is adapted to provide at least the operational mode. Would other modes be available, the choice of such modes may especially be executed via a user interface, though other options, like executing a mode in dependence of a sensor signal or a (time) scheme, may also be possible. The operation mode may in embodiments also refer to a system, or apparatus, or device, that can only operate in a single operation mode (i.e. “on”, without further tunability).

In embodiments, the system, especially the control system, may have a first operational mode and a second operational mode. The term “operational mode” may also be indicated as “controlling mode”.

In embodiments, in the first operational mode one or more of the one or more light sources may be configured to generate first light source light, especially first light source light having one or more wavelengths selected from the range of 100-380 nm. The first light source light may herein also be referred to as disinfecting light. Further, the one or more wavelengths may be selected suitable to disinfect the closeable space.

Especially, in embodiments the first light source light may have at least 80%, such as at least 90%, especially at least 95%, such as at least 99%, including 100%, of its spectral power in the wavelength range of 100-2000 nm in the wavelength range of 100-380 nm, especially in the wavelength range of 190-380 nm, such as in the wavelength range of 200-315 nm, especially in the wavelength range of 200-280 nm, such as in the wavelength range of 230-280 nm.

Phrases like “light source light may have at least 80% of its spectral power in the wavelength range of 100-2000 nm in the wavelength range of 100-380 nm”, and similar phrases, may indicate that the light source light has a first spectral power over the wavelength of 100-2000, and a second spectral power over the wavelength range of 100-380 nm. The latter is also included in the former. The percentage of the second spectral power relative to the first spectral power is the indicated percentage, in this example 80%. Hence, e.g. assuming 1000 Watt in the range of 100-2000 nm, including 800 Watt in the range of 100-380 nm, and 200 Watt in the range of 380-2000 nm, would lead to a percentage of 80% of its spectral power in the wavelength range of 100-2000 nm in the wavelength range of 100-380 nm.

In further embodiments, the first light source light may have at least 80% such as at least 90%, especially at least 95%, such as at least 99%, including 100%, of its spectral power in the wavelength range of 100-800 nm in the wavelength range of 100-380 nm, especially in the wavelength range of 190-380 nm, such as in the wavelength range of 200-315 nm, especially in the wavelength range of 200-280 nm, such as in the wavelength range of 230-280 nm.

In further embodiments, the first light source light may have a centroid wavelength in the wavelength range of 190-380 nm, such as in the wavelength range of 200-315 nm, especially in the wavelength range of 200-280 nm, such as in the wavelength range of 230-280 nm.

In further embodiments, in the second operational mode one or more of the one or more light sources may be configured to generate second light source light having a color point in the visible wavelength range, especially having a centroid wavelength selected from the range of 400-490 nm.

Especially, in embodiments the second light source light may have at least 80% of its spectral power in the wavelength range of 100-2000 nm in the wavelength range of 400-490 nm, especially in the wavelength range of 400-450 nm, such as in the range of 405-440 nm, especially in the range of 410-430 nm.

In further embodiments, the second light source light may have at least 80% such as at least 90%, especially at least 95%, such as at least 99%, including 100%, of its spectral power in the wavelength range of 100-800 nm in the wavelength range of 400-490 nm, especially in the wavelength range of 400-450 nm, such as in the range of 405-440 nm, especially in the range of 410-430 nm.

In further embodiments, the first light source light may have a centroid wavelength in the wavelength range of 400-490 nm, especially in the wavelength range of 400-450 nm, such as in the range of 405-440 nm, especially in the range of 410-430 nm.

The second light source light may herein also be referred to as indicator light. The color point, especially the centroid wavelength, may in embodiments be selected to convey a safe and clean feeling to a user.

The first light source light and the second light source light may especially be provided in and/or to the closeable space. Hence, (at least part of) the one or more light sources may be arranged in the closeable space. Further, the one or more light sources may be configured to provide the (respective) first and/or second light source light to the closeable space. Especially, all the light sources of the one or more light sources are configured in the closeable space.

In embodiments, the system may further comprise a sensor, especially in embodiments a sensor selected from the group comprising a heat sensor, a motion sensor, a touch sensor, and a light sensor, especially from the group comprising a motion sensor and a touch sensor. Other sensors may also be possible. A sensor based on a change in resistance, like e.g. a potentiometer, or a slide potentiometer, may also be possible.

In further embodiments, the sensor may be configured to detect a state or a change in state of the closeable space. In particular, the sensor may be configured to detect whether the closeable space is in an open state or a closed state. In further embodiments, the sensor may be configured to detect whether the closeable space is in a partially open state (also see below), or especially to determine an extent of openness of the closeable space. Alternatively or additionally, the sensor may be configured to detect a change in state of the closeable space. For instance, when approaching or pushing a touch button, or other button that may sense touch or close proximity of each a finger, the system may already know the change of state before the change of state is executed. Likewise, this may apply to soft close/soft opening systems, wherein a e.g. a force in a closing direction of a closing element, when the closeable space is already closed, will result in an opening of the closeable space.

Hence, the system may comprise a sensor, wherein the sensor may in embodiments be configured to determine a parameter related to a state or a change in state of the closeable space, especially related to the openness of the closeable space.

The sensor may further be configured to provide a related sensor signal to the control system, i.e., the sensor may be configured to determine a parameter related to a state or a change in state of the closeable space and to provide a related sensor signal to the control system.

The term “related signal” may herein refer to a signal that is related to the detected parameter. In particular, the related signal may comprise raw and/or processed data related to the (detected) parameter, such as raw and/or processed data related to the openness of the closeable space.

In embodiments, the system (for disinfecting at least part of a closeable space) may comprise a control system. The control system may be configured to control the system, especially the one or more light sources and/or the sensor, such as especially at least the one or more light sources.

The term “controlling” and similar terms herein may especially refer at least to determining the behavior or supervising the running of an element. Hence, herein “controlling” and similar terms may e.g. refer to imposing behavior to the element (determining the behavior or supervising the running of an element), etc., such as e.g. measuring, displaying, actuating, opening, shifting, changing temperature, etc. . . . Beyond that, the term “controlling” and similar terms may additionally include monitoring. Hence, the term “controlling” and similar terms may include imposing behavior on an element and also imposing behavior on an element and monitoring the element. The controlling of the element can be done with a control system. The control system and the element may thus at least temporarily, or permanently, functionally be coupled. The element may comprise the control system. In embodiments, the control system and the element may not be physically coupled. Control can be done via wired and/or wireless control. The term “control system” may also refer to a plurality of different control systems, which especially are functionally coupled, and of which e.g. one control system may be a control system and one or more others may be slave control systems.

In particular, the control system may be configured to select an operational mode of the system i.e., the control system may select (between) the first operational mode and the second operational mode (and the third operational mode; see below).

In embodiments, the control system may be configured to select the first operational mode during a first time period when the closeable space is configured in a closed state.

The term “closed state” may herein refer to a state wherein the closeable space is essentially closed off. In particular, the term “closed state” may herein refer to a state wherein light generated in the closeable space, especially first light source light generated in the closeable space, essentially may not exit the closeable space. Hence, in the close state light generated in the closeable space, especially second light source light generated in the closeable space, may not exit the closeable space and may not be observed by a user (external from the closable space) observing the closeable space.

In embodiments, the first time period may be selected suitable to disinfect the closeable space. It will be clear to the person skilled in the art, that the amount of time suitable to disinfect a closeable space may depend on various factors, such as the size of the closeable space, the intensity of the disinfecting light, and the microbes and/or viruses that may be present. For instance, in embodiments, the first time period may be selected from the range of 5-300 minutes, such as from the range of 10-180 minutes, such as from the range of 15-60 minutes.

In further embodiments, the control system may be configured to select the second operational mode during a second time period when the closeable space is configured in the open state.

The term “open state” may herein refer to a state wherein the closeable space is essentially open. In particular, the term “open state” may herein refer to a state wherein light generated in the closeable space, especially second light source light generated in the closeable space, may exit the closeable space and may be observed by a user (external from the closable space) observing (or especially using) the closeable space. The term “open state” may herein especially refer to a fully open state, but may also refer to a partially open state. Hence, herein, also the terms “fully open state” and “partially open state” may be used to, respectively, refer to a closeable space (essentially) being fully open or partially open.

In embodiments, the second time period may be selected suitable to convey a clean and safe feeling to a user. In particular, if the second time period is too short, the user may not notice the second light source light, but if the second time period is too long, the user may worry about the disinfecting light not having been switched off. Hence, in embodiments, the second time period may be selected from the range of 0.05-10 seconds, such as from the range of 0.1-5 seconds, especially from the range of 0.5-3 seconds. In further embodiments, the second time period may be >0 seconds, especially ≥0.01 seconds, such as ≥0.05 seconds, especially ≥0.1 seconds, such as ≥0.5 seconds, especially ≥1 second.

It will be clear to the person skilled in the art that different values of the second time period may be selected for different types of closeable spaces. For example, a larger value, such as from the range of 0.1-10 seconds, especially from the range of 2-10 seconds, may be selected for a closeable space opened via a (heavy) door, whereas a smaller value, such as from the range of 0.1-3 seconds may be selected for applications wherein the closeable space may be opened relatively quickly, such as for a kitchen drawer.

In embodiments, the second light source light may have a second light intensity, especially during at least part of the second time period. In further embodiments, the second light source light may have a second luminous flux, wherein the second luminous flux may be at least 30 lm, especially at least 70 lm, such as at least 100 lm, especially at least 150 lm, such as at least 200 lm.

In further embodiments, the second luminous flux may be at most 1000 lm, such as at most 700 lm, especially at most 500 lm, such as at most 300 lm, especially at most 200 lm.

In further embodiments, the control system may be configured to alternate between selecting the first operational mode and the second operational mode as the closeable space may alternate between being in a closed state and an open state. Especially, the control system may select the first operational mode and/or the second operational mode on the basis of the related signal from the sensor.

In embodiments, the system, especially the control system, may be configured to select the second operational mode while or after changing from the closed state to the open state, especially while or after the closeable space changed from the closed state to the open state. In particular, as a user opens the closeable space, the system, especially the control system, may (instantly) switch to the second operational mode, such that the second light source light is (instantly) provided to the user.

Further, in embodiments, the control system may be configured to reduce a first light intensity of the first light source light when the closeable space changes from a closed state to an open state, especially reduce the first light intensity to at most 10%, such as at most 1%, of the first light intensity of the first light source light during the first time period, i.e., the control system may be configured to dim the first light source light when the closeable space changes from a closed state to an open state, especially dim the first light source light to at most 10%, such as at most 5%, especially at most 1%, including (down to) 0%. In general, the control system may be configured to switch the first light source light off (or to keep it off) when the closeable space changes from the closed state to the open state, i.e., the control system may be configured to switch off the first light source light when the closeable space changes from the closed state to the (partially) open state. Further, in general, the control system may be configured to prevent the one or more light sources from providing first light source light while the closeable space is in the open state, especially when the system is in the second or third operational mode.

The second light source light and the third light source light (see below) may primarily be intended for the user rather than for disinfection. Hence, the second light source light and the third light source light may essentially be off when the closeable space is in the closed state, especially when the system is in the first operational mode.

Hence, in embodiments, the control system may be configured to reduce a second light intensity of the second light source light when the closeable space changes from an open state to a closed state, especially reduce the second light intensity to at most 10%, such as at most 1%, of the second light intensity of the second light source light during the second operational mode, especially during the second time period, i.e., the control system may be configured to dim the second light source light when the closeable space changes from an open state to an closed state, especially dim the second light source light to at most 10%, such as at most 5%, especially at most 1%, including (down to) 0%.

Similarly, in embodiments, the control system may be configured to reduce a third light intensity of the third light source light when the closeable space changes from an open state to a closed state, especially reduce the third light intensity to at most 10%, such as at most 1%, of the third light intensity of the third light source light during the third operational mode, especially during the third time period, i.e., the control system may be configured to dim the third light source light when the closeable space changes from an open state to an closed state, especially dim the third light source light to at most 10%, such as at most 5%, especially at most 1%, including (down to) 0%.

As the user may be incapable of visually determining whether the first light source light is indeed off, the user may be comforted by the second light source light gradually switching off.

Hence, in embodiments, the control system may be configured to gradually decrease the second light intensity over at least part of the second time period. Hence, during the second operational mode, the second light source light may be provided at an initial second light intensity I₂₁ at the start of the second time period and at a final second light intensity I_{2F} at the end of the second time period, wherein I_{2F}<I₂₁, such as I_{2F}≤0.5*I₂₁, especially I₂F≤0.1*I₂₁. In particular, the final second light intensity may essentially be 0. In embodiments, the gradual decrease is linear in time. In alternative embodiments, the decrease is logarithmic in time.

The gradual decrease of the second light intensity may also be provided at a delay. Thereby, the user may be more likely to note the second light source light, such as in a bright environment, and the user may still observe the gradual decrease of the second light intensity. Hence, in further embodiments, the control system may be configured to gradually decrease the second light intensity over a final part of the second time period, wherein the final part is at least 0.5 seconds, such as at least 1 second, especially at least 2 seconds. Similarly, in further embodiments, the control system may be configured to keep the second light intensity (essentially) constant over an initial part of the second time period, wherein the initial part is at least 0.5 seconds, such as at least 1 second, especially at least 2 seconds. In further embodiments, the second time period may consist of the initial part and the final part.

In embodiment, the sensor may be configured to detect a parameter related to the opening and/or openness of the closeable space, and especially to provide a related signal to the control system. In particular, in embodiments, the sensor may be configured to detect whether the closeable space is in a closed state or in an open state and to provide a related sensor signal to the control system. For instance, the sensor may comprise a touch-based sensor (see also above), such as a pressure sensor, configured to detect whether a moveable element for opening and closing the closeable space is configured in a closing position.

However, in further embodiments, the sensor may (further) be configured to detect that the closeable space may soon be opened. For instance, the sensor may be functionally coupled to a handle for opening the closeable space, wherein the sensor may detect the handle being touched (by a user), and wherein the sensor may provide a related signal to the control system. Similarly, the sensor may comprise one or more of a motion sensor, an audio sensor or a heat sensor, and may detect a presence near the closeable space and provide a related signal to the control system. In such embodiments, the control system may estimate whether the closeable space is likely to be opened, such as based on historical data, and may be configured to switch off the first light source light if the likelihood of the closeable space to be opened is above a predetermined threshold. For instance, if the handle corresponding to the closeable space is touched, it may be fairly likely that the closeable space is going to be opened, and the control system may be configured to switch off the first light source light (as a precautionary measure). However, if the closeable space is for instance comprised by a rarely opened kitchen drawer, then a presence near the kitchen drawer, such as detected by a motion sensor, may not prompt the control system to switch off the first light source light.

Also, combinations of the aforementioned may be applied, i.e., the sensor may both detect whether the closeable space is in an open state or in a closed state, as well as detect one or more parameters related to the opening of the closeable space. The term “sensor” may herein also refer to a plurality of sensors.

In embodiments, the sensor may especially be selected from the group comprising a heat sensor, a motion sensor, a touch sensor, and a light sensor.

The second light source light may be indicative of the cleanness (or the “cleaning”) of the closeable space. However, if the closeable space is repeatedly opened, it may be that insufficient time has passed for the first light source light to clean the closeable space. Hence, in embodiments, the control system may be configured to select the second operational mode only if the system has been in the first operational mode for a predetermined consecutive period, especially wherein the predetermined consecutive period is at least 2 minutes, such as at least 3 minutes, especially at least 5 minutes, such as at least 8 minutes, especially at least 10 minutes, such as at least 15 minutes, especially at least 20 minutes, such as at least 30 minutes. In embodiments, the predetermined consecutive period may be at least part of the first time period, such as at least half of the first time period, especially at least three-quarter of the first time period, such as essentially equal to the first time period. Hence, the predetermined consecutive period may be the first time period. In such embodiments, a user will be aware of whether sufficient cleaning has taken place, which may provide additional comfort to the user when the second light source light switches on when the user opens the closeable space.

Hence, in embodiments, the system may be configured to only provide the second light source light when the first light source light has been provided for a predetermined duration, i.e., when sufficient disinfection has taken place. Hence, the second light source light may indicate both that the closeable space is (now) safe to use, and that disinfection has taken place.

In further embodiments, the control system may be configured to select the second operational mode only if a predetermined radiant exposure of first light source light has been provided to (a surface in) the closeable space. In further embodiments, the predetermined radiant exposure may be selected from the range of 10-100 J/cm² within an 8-hour time interval, such as ≤50 J/m² within an 8-hour time interval, especially ≤40 J/m² within an 8-hour time interval, especially at most 30 J/m² within an 8-hour time interval.

Hence, in embodiments, the first operational mode may comprise (the one or more of the one more light sources) providing a radiant exposure of first light source light to (a surface in) the closeable space, wherein the radiant exposure may be selected from the range of 10-100 J/m² within an 8-hour time interval, such as ≤50 J/m² within an 8-hour time interval, especially ≤40 J/m² within an 8-hour time interval, especially at most 30 J/m² within an 8-hour time interval.

In further embodiments, the first operational mode may comprise (the one or more of the one more light sources) providing the first light source light at a first light intensity to (a surface in) the closeable space for a first exposure time.

In further embodiments, the control system may be configured to select the second operational mode only if the first light source light has been provided to (a surface in) the closeable space at a (predetermined) first light intensity for a (predetermined) first exposure time.

In embodiments, the first light intensity may be at least 0.01 mW/m², such as at least 0.1 mW/m², especially at least 0.5 mW/m². In further embodiments, the first light intensity may be at most 20 mW/m², such as at most 10 mW/m², especially at most 5 mW/m², such as at most 1 mW/m².

In further embodiments, the first exposure time may be at least 1 minute, such as at least 5 minutes, especially at least 10 minutes, such as at least 1 hour, especially at least 4 hours, such as at least 8 hours. In further embodiments, the first exposure time may be at most 36 hours, such as at most 24 hours, especially at most 12 hours.

In some circumstances, the closeable space may remain closed for extensive periods of time. For instance, the closeable space may be rarely used, or the user may be on holidays. As anti-microbial and/or anti-viral measures may generally not eliminate all microbes and/or viruses, the microbes and/or viruses may again accumulate over time if the first light source light is only used once (directly) after the closeable space is closed. However, continuous use of the first light source light may be undesirable in view of, for instance, energy consumption. Hence, in embodiments, the control system may be configured to, during the first operational mode, provide the first light source light, especially for the first time period, according to a predefined temporal pattern, such as every 4 hours, especially every 6 hours, such as every 8 hours, especially every 12 hours, such as at least once daily, especially at least once every two days, such as at least once every week.

In embodiments, the first operational mode may comprise (the one or more of the one or more light sources) providing the first light source light for a consecutive period of at least 2 minutes, such as at least 3 minutes, especially at least 5 minutes, such as at least 8 minutes, especially at least 10 minutes, such as at least 15 minutes, especially at least 20 minutes, such as at least 30 minutes.

In further embodiments, the consecutive period may be selected in view of the predefined temporal pattern (or vice versa). For example, the first light source light may beneficially be provided at a high interval with relatively short consecutive periods, or may beneficially be applied at a low interval with relatively long consecutive periods. For example, a consecutive period of at least 30 min may be selected for applications wherein typically a limited number of disinfection steps are executed (or “needed”) per day.

In embodiments, the disinfecting light, especially the first light source light, may especially comprise ultraviolet (UV) radiation, i.e., the first light source light may comprise a wavelength selected from the ultraviolet wavelength range. The ultraviolet wavelength range is defined as light in a wavelength range from 100 to 380 nm and can be divided into different types of UV light/UV wavelength ranges (Table 1). Different UV wavelengths of radiation may have different properties and thus may have different compatibility with human presence and may have different effects when used for disinfection (Table 1).

TABLE 1
Properties of different types of UV wavelength light

| | | | (Relative) | | | | |
|---|---|---|---|---|---|---|---|
| | | | sterilization | | | | |
| | Short | Wavelength | effectiveness | | Safe | Vitamin D | Ozone |
| Name | name | (nm) | Bacteria | Viruses | Radiation | generation | generation |
| Violet | V | 380-420 | +/− | − | + | | |
| Ultra-violet A | UV-A | 315-380 | + | − | + | | |
| Ultra-violet B | UV-B | 280-315 | + | +/− | +/− | + | |
| Near ultra- | Near | 230-280 | + | + | − | | |
| violet C | UV-C | | | | | | |
| Far ultra-violet | Far UV | 190-230 | + | + | + | | +/− |
| Extreme | Extreme | 100-190 | + | + | − | | + |
| ultra-violet C | UV-C | | | | | | |

Each UV type/wavelength range may have different benefits and/or drawbacks. Relevant aspects may be (relative) sterilization effectiveness, safety (regarding radiation), and ozone production (as result of its radiation). Depending on an application a specific type of UV light or a specific combination of UV light types may be selected and provides superior performance over other types of UV light. UV-A may be (relatively) safe and may kill bacteria, but may be less effective in killing viruses. UV-B may be (relatively) safe when a low dose (i.e. low exposure time and/or low intensity) is used, may kill bacteria, and may be moderately effective in killing viruses. UV-B may also have the additional benefit that it can be used effectively in the production of vitamin D in a skin of a person or animal. Near UV-C may be relatively unsafe, but may effectively kill bacteria and viruses. Far UV may also be effective in killing bacteria and viruses, but may be (relatively to other UV-C wavelength ranges) (rather) safe. Far-UV light may generate some ozone which may be harmful for human beings and animals. Extreme UV-C may also be effective in killing bacteria and viruses, but may be relatively unsafe. Extreme UV-C may generate ozone which may be undesired when exposed to human beings or animals. In some application ozone may be desired and may contribute to disinfection, but then its shielding from humans and animals may be desired. Hence, in the table “+” for ozone production especially implies that ozone is produced which may be useful for disinfection applications, but may be harmful for humans/animals when they are exposed to it. Hence, in many applications this “+” may actually be undesired while in others, it may be desired.

Hence, in embodiments, the first light source light may comprise a wavelength in the UV-A range. In further embodiments, the first light source light may comprise a wavelength in the UV-B range. In further embodiments, the first light source light may comprise a wavelength in the Near UV-C range. In further embodiments, the first light source light may comprise a wavelength in the Far UV range. In further embodiments, the first light source light may comprise a wavelength in the extreme UV-C range.

The Near UV-C, the Far UV and the extreme UV-C ranges may herein also collectively be referred to as the UV-C range. Hence, in embodiments, the first light source light may comprise a wavelength in the UV-C range.

In embodiments, the first light source light may have one or more wavelengths selected from the UV-B range and/or from the UV-C range, especially from the range of 100-315 nm. As indicated above, first light source light having these wavelengths may be effective against bacteria and moderately effective against viruses.

In embodiments, the first light source light may have one or more wavelengths selected from the UV-C range, especially from the range of 100-280 nm. As indicated above, first light source light having these wavelengths may be effective against bacteria and viruses.

Given that the first light source light may (essentially) only be provided during the first operational mode, the first light source light may in embodiments comprise one or more wavelengths selected from the Near UV-C and/or the extreme UV-C ranges. However, as a precaution it may be preferable to select the wavelengths of the first light source light to be safe for humans. Hence, in further embodiments, the first light source light may have one or more wavelengths from the Far UV and the UV-B ranges, especially from the Far UV range, especially wherein an intensity of the first light source light in the Far UV and the UV-B ranges may be at least 10 times higher than the intensity of the first light source light in other UV ranges.

In further embodiments, the first light source light may have one or more wavelengths selected from the range of 100-190 nm and/or from the range of 230-315 nm.

In embodiments, the system, especially the control system, may have a third operational mode. The control system may especially be configured to select the third operational mode during a third time period when the closeable space is configured in the open state.

In the third operational mode, one or more of the one or more light sources may be configured to generate third light source light. The third light source light may especially be for the illumination of the closeable space. Hence, the third light source light may especially be white light. In embodiments, the third light source light may have a correlated color temperature selected from the range of 1800-20000 K, especially from the range of 1800-6500 K, such as selected from the range of 2000-5000 k.

The first light source light may be provided by one on or more light sources. In specific embodiments, the first light source light may in embodiments be provided by a plurality of essentially the same light sources, such as LEDs from the same bin. In other embodiments, however, the first light source light may be provided by a plurality of different light sources, such as different LEDs (from different bins).

The second light source light may be provided by one on or more light sources. In specific embodiments, the second light source light may in embodiments be provided by a plurality of essentially the same light sources, such as LEDs from the same bin. In other embodiments, however, the second light source light may be provided by a plurality of different light sources, such as different LEDs (from different bins).

The third light source light may be provided by one on or more light sources. In specific embodiments, the third light source light may in embodiments be provided by a plurality of essentially the same light sources, such as LEDs from the same bin. In other embodiments, however, the third light source light may be provided by a plurality of different light sources, such as different LEDs (from different bins).

Hence, the system may be configured such that the system, especially one or more of the one or more light sources, provides second light source light as the closeable space opens, to signify that the space is disinfected, and to provide third light source light when the closeable space is open to facilitate use of the closeable space.

In embodiments, the one or more light sources may be divided into first light sources configured to provide first light source light, second light sources configured to provide second light source light, and third light sources configured to provide third light source light.

In further embodiments, at least part of the one or more light sources may be configured to provide two or more, especially each, of the first light source light, the second light source light and the third light source light. Such embodiments may be particularly beneficial as the total number of light sources may be reduced.

In further embodiments, at least part of the one or more light sources may be configured to provide the first light source light and at least one of the second light source light and the third light source light. Such embodiments may be particularly beneficial as the user may note that the light sources for disinfecting are still functioning when the user observes these light sources providing a different type of light source light.

Hence, one or more light sources may comprise a first light emitting device to generate, in operation, the first light source light, and a second light emitting device providing, in operation, the second light source light. In particular, a single light source may comprise the first and second light emitting devices, especially each of a plurality of light sources of the one or more light sources, such as all of the one or more light sources, may comprise a first and a second light emitting device. In further embodiments, a first light source (of the one or more light sources) may comprise the first light emitting device while a second light source (of the one or more light sources) may comprise the second light emitting device.

The term “first light source” may especially refer to a light source (of the one or more light sources) configured to provide (at least) the first light source light. Similarly, the term “second light source” (or “third light source”) may herein refer to a light source configured to provide (at least) the second (or third) light source light. Hence, the system may comprise one or more first light sources, one or more second light sources, and/or one or more third light sources.

In embodiments, one or more light sources (of the one or more light sources) may comprise a third light emitting device configured to provide third light source light. In further embodiments, each light source (of the one or more light sources) may comprise the first, second and third light emitting devices.

In further embodiments, a first light source (of the one or more light sources) may comprise the first light emitting device and a second light source (of the one or more light sources) may comprise the second and third light emitting devices. In further embodiments, first light source (of the one or more light sources) may comprise the first light emitting device, and a second light source (of the one or more light sources) may comprise the second light emitting device, and a third light source (of the one or more light sources) may comprise the third light emitting device.

In embodiments, the first light source and the second light source may be arranged at a distance, especially at a shortest distance, of at least 5 cm, such as of at least 10 cm, especially of at least 15 cm. Especially, each first light source may be arranged at a (shortest) distance of at least 5 cm, such as of at least 10 cm, especially of at least 15 cm from each second light source.

In further embodiments, the first light source, the second light source and the third light source may (each) be arranged at (respective) shortest distances of at least 5 cm, such as of at least 10 cm, especially at least 15 cm.

In further embodiments, each light source (of the one or more light sources) may be arranged at a (shortest) distance of at least 5 cm, such as of at least 10 cm, especially of at least 15 cm from each other light source (of the one or more light sources).

In further embodiments, the closeable space may be (at least partially) defined by a plurality of surfaces. In such embodiments, the one or more first light sources may especially be arranged at a different surface of the plurality of surfaces than the one or more second light sources. Similarly, in embodiments, the one or more first light sources (or second light sources) may be arranged at a different surface of the plurality of surfaces than the one or more third light sources.

In embodiments, the system, especially the control system, may be configured to select the third operational mode when the closeable space is in the open state.

In further embodiments, the system, especially the control system, may be configured to select the third operational mode when the closeable space is in the open state for at least 1 second, such as for at least 2 seconds, especially for at least 3 seconds. Hence, the system, especially the control system, may be configured to select the third operational mode after a delay. In further embodiments, the system, especially the control system, may be configured to select the third operational mode when the closeable space is in the open state for at most 7 seconds, such as for at most 5 seconds, especially for at most 3 seconds, such as at most 2 seconds. Hence, the system, especially the control system, may be configured to select the third operational mode soon after the opening of the closeable space.

In embodiments, the second operational mode and the third operational mode may be temporally separated, especially wherein the third operational mode is temporally arranged after the second operational mode. Hence, upon opening of the closeable space, initially the second light source light may be provided, and subsequently the third light source light may be provided. In further embodiments, the third operational mode may (directly) follow the second operational mode, especially wherein the second operational mode and the third operational mode are temporally separated by less than 1 second, such as by less than 0.5 seconds, especially less than 0.1 seconds.

In further embodiments, the second operational mode and the third operational mode may partially overlap in time. In particular, the system, especially the control system, may be configured to gradually change from the second operational mode to the third operational mode. Hence, the second operational mode and the third operational mode may at least partially (temporally) overlap during a transitional time period. Especially, the system, especially the control system, may be configured to gradually decrease the second light intensity of the second light source light during the transitional time period, and/or to gradually increase a third light intensity of the third light source light during the transitional time period.

In further embodiments, the second time period and the third time period may temporally overlap during a transitional time period, especially wherein the control system is configured to gradually decrease the second light source light and/or to gradually increase the third light source light during the transitional time period.

In further embodiments, the control system may be configured to gradually reduce an intensity of the second light source light during at least part of the second time period. In further embodiments, the control system may be configured to gradually increase an intensity of the third light source light during at least part of a third time period.

In further embodiments, the control system may be configured to gradually decrease (the second light intensity of) the second light source light and to gradually increase (the third light intensity of) the third light source light during a transitional time period, wherein the transitional time period comprises at least part of the second time period and at least part of the third time period.

In embodiments, the first time period and the second time period may be temporally separated. Similarly, in further embodiments, the first time period and the third time period may be temporally separated.

In embodiments, the sensor may be configured to determine whether the closeable space is in a partially open state, and especially to provide a related sensor signal to the control system. For instance, the sensor may comprise a motion sensor and/or a pressure sensor and/or a light sensor to determine whether the closeable space is in a partially open state.

The term “partially open state” may herein refer to a state between a closed state and a fully open state. For instance, with respect to a drawer, the drawer may be arranged at a first (closed) (end) position P1, and may be configured to be moveable up to a movement limit L1 from the first position P1, wherein during use the drawer may have a movement distance d1 to the first position P1, wherein the closeable space is (considered to be) in the partially open state when 0.03*L1≤d1≤0.8*L1. It will be clear to the person skilled in the art that different thresholds may apply to different types of closeable spaces. For instance, a door may, for example, be moveable 150° along its hinges, but may generally already be regarded fully open at about 90°, while a drawer may, for example, generally only be considered fully open when the back of the drawer is in view of a user.

In embodiments, the control system may be configured to reduce the second light intensity when the closeable space changes from the partially open state to the (fully) open state, especially to reduce the second light intensity to at most 10%, such as at most 1%, of the second light intensity (when the closeable space is) in the partially open state, i.e., the control system may be configured to dim the second light source light when the closeable space changes from the partially open state to the open state, especially dim the second light source light to at most 10%, such as at most 5%, especially at most 1%, including (down to) 0%.

Hence, the system, especially the control system, may be configured to switch from the second operational mode to the third operational mode based on a duration and/or based on an extent at which the closeable space is opened. In particular, if the closeable space is only opened slightly, the second operational mode may last longer than when the closeable space is immediately fully opened.

In a further aspect, the invention may provide a structure comprising the system according to the invention. Especially, the structure may define the closeable space, and the structure may especially comprise a moveable element for opening and closing the closeable space. In embodiments, the moveable element may be selected from the group comprising a lid, a door, and a drawer. The movable element may also be indicated as “closing element”.

In further embodiments, the sensor may be configured to determine a state or a change in state of the closeable space based on a position of the moveable element, especially to determine whether the closeable space is in the closed state, (the partially open state) or in the open state based on a position of the moveable element.

Hence, the invention may provide a structure defining the closeable space. In embodiments, the structure may, for example, be selected from the group comprising a walled space, especially a house, a box, a container, a cabinet, and a vehicle, especially a car. The structure may comprise a plurality of structural components together defining the closeable space. For instance, a walled space may comprise one or more walls, a floor, a ceiling, and optionally one or more windows and/or one or more doors defining the closeable space. Similarly, a cabinet may comprise a frame and one or more drawers together defining a closeable space.

In embodiments, the closeable space may be selected from the group of drawers, appliance cabinets, specialty cabinets, corner cabinets, tall cabinets, base cabinets, and top cabinets.

The term “moveable element” may herein refer to any element suitable to close the closeable space. For instance, the moveable element may be a (car) door, or it may be a drawer. In particular, the moveable element may be arranged in a plurality of positions, wherein at least one first position P1 of the plurality of positions corresponds to the closeable space being in a closed state.

In embodiments, the sensor may especially be configured to detect the position of the moveable element, especially at which of the plurality of positions the moveable element is (currently) arranged.

In further embodiments, the moveable element may be arranged (or: “arrangeable”) at a first (closed) (end) position P1, and the moveable element may be configured moveable up to a movement limit L1 from the first position P1. Especially, during use the moveable element may have a movement distance d1 to the first position P1, especially wherein d1≤L1.

The term “movement limit” may herein refer to a length, but may, for instance, also refer to a rotation depending on the relevant unit for the moveable element. For example, with respect to a drawer a length may be most appropriate, while for a (car) door a rotation, especially a rotation angle, may be most appropriate. Similarly, the term “movement distance” may herein refer to a length, but also to an angle. In particular, the movement distance d1 is in the same unit as the movement limit L1.

In embodiments, the sensor may be configured to determine the movement distance d1 and to provide a related sensor signal to the control system.

In further embodiments, the control system may determine whether the closeable space is in the closed state or the open state depending on the distance d1. In particular, in embodiments, (the control system may be configured to determine that) the system is in the closed state when d1≤0.1*L1, such as ≤0.05*L1, especially ≤0.03*L1, such as ≤0.02*L1, especially ≤0.01*L1, including 0. Further, in embodiments, (the control system may be configured to determine that) the system is in the open state when d1 is not in the closed state, especially wherein d1>0.01*L1, such as >0.02*L1, especially >0.03*L1, such as >0.05*L1, especially >0.1*L1.

As also indicated above, the system, especially the control system may, in embodiments, distinguish between a partially open state and a (fully) open state. Hence, in further embodiments, (the control system may be configured to determine that) the closeable space is in the fully open state when d1 especially wherein d1≥0.5*L1, such as ≥0.6*L1, especially ≥0.7*L1, such as ≥0.8*L1, especially ≥0.9*L1, such as ≥0.95*L1, such as L1.

Further, in such embodiments, (the control system may be configured to determine that) the closeable space is in the partially open state when it is not in the closed state nor in the open state. For instance, in embodiments, (the system may be configured to determine that) the closeable space is in the partially open state when 0.02*L1<d1<0.9*L1, such as when 0.03*L1<d1<0.8*L1, especially when 0.05*L1<d1<0.7*L1.

Hence, in embodiments, the closeable space may, for instance, be in the closed state when d1<0.03*L1, in the partially open state when 0.03*L1<d1<0.8*L1; and in the open state when 0.8*L1≤ d1.

In a further aspect, the invention may provide a kit of parts, especially wherein the kit of parts comprises one or more light sources, a control system, and a sensor, and optionally a manual. In embodiments the control system may be configured to select a first operational mode and a second operation mode for the one or more light sources, especially wherein in the first operational mode one or more of the one or more light sources are configured to generate first light source light having one or more wavelengths selected from the range of 100-380 nm, and especially wherein in the second operational mode one or more of the one or more light sources are configured to generate second light source light having a color point in the visible wavelength range. In embodiments, the second light source light may have a centroid wavelength selected from the range of 400-495 nm. In further embodiments, the sensor may be selected from the group of a heat sensor, a motion sensor, a touch sensor, and a light sensor.

In embodiments, the kit of parts may be configured to provide the system of the invention.

In further embodiments, the manual may comprise instructions suitable to inform a user how to provide the system of the invention using the kit of parts.

In further embodiments, the manual may comprise instructions on how to arrange the one or more light sources in the closeable space.

In a specific embodiment, the light source may comprise a solid state LED light source (such as a LED or laser diode).

In a further aspect, the invention may comprise a drawer comprising and/or functionally coupled to the system of the invention. In particular, the one or more light sources may be arranged in the drawer, and the sensor may be arranged functionally coupled to the drawer. In embodiments, the sensor and/or the control system may be arranged in the drawer. In further embodiments, the sensor and/or the control system may be attached to the drawer, especially at an outside surface of the drawer. In further embodiments, the sensor and/or the control system may be arranged external of the drawer in a cabinet comprising the drawer.

The term “light source” may also relate to a plurality of light sources, such as 2-200 (solid state) LED light sources. Hence, the term LED may also refer to a plurality of LEDs.

The term white light herein, is known to the person skilled in the art. It especially relates to light having a correlated color temperature (CCT) between about 2000 and 20000 K, especially 2700-20000 K, for general lighting especially in the range of about 2700 K and 6500 K, and for backlighting purposes especially in the range of about 7000 K and 20000 K, and especially within about 15 SDCM (standard deviation of color matching) from the BBL (black body locus), especially within about 10 SDCM from the BBL, even more especially within about 5 SDCM from the BBL.

In an embodiment, the light source may also provide third light source light having a correlated color temperature (CCT) between about 5000 and 20000 K, e.g. direct phosphor converted LEDs (blue light emitting diode with thin layer of phosphor for e.g. obtaining of 10000 K). Hence, in a specific embodiment the light source is configured to provide light source light with a correlated color temperature in the range of 5000-20000 K, even more especially in the range of 6000-20000 K, such as 8000-20000 K. An advantage of the relative high color temperature may be that there may be a relative high blue component in the light source light.

The terms “violet light” or “violet emission” especially relates to light having a wavelength in the range of about 380-440 nm. The terms “blue light” or “blue emission” especially relates to light having a wavelength in the range of about 440-495 nm (including some violet and cyan hues). The terms “green light” or “green emission” especially relate to light having a wavelength in the range of about 495-570 nm. The terms “yellow light” or “yellow emission” especially relate to light having a wavelength in the range of about 570-590 nm. The terms “orange light” or “orange emission” especially relate to light having a wavelength in the range of about 590-620 nm. The terms “red light” or “red emission” especially relate to light having a wavelength in the range of about 620-780 nm. The term “pink light” or “pink emission” refers to light having a blue and a red component. The terms “visible”, “visible light” or “visible emission” refer to light having a wavelength in the range of about 380-780 nm.

The embodiments described herein are not limited to a single aspect of the invention. For example, an embodiment describing the system may, for example, further relate to the structure, or especially to the kit of parts. Similarly, an embodiment of the structure describing an operation of the system may further relate to embodiments of the system.

The system may be part of or may be applied in e.g. a room, a fridge, a freezer, a washing machine, a dish washer, a cupboard, a drawer, a microwave, an oven, a cabinet, a car, a bus, a container, a containment, a hospital (room/cupboard), a restaurant (kitchen/cupboard), et cetera.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIG. 1A-B schematically depict embodiments of the system of the invention.

FIG. 2 schematically depicts a further embodiment of the system of the invention. The schematic drawings are not necessarily on scale.

### DETAILED DESCRIPTION OF THE EMBODIMENTS

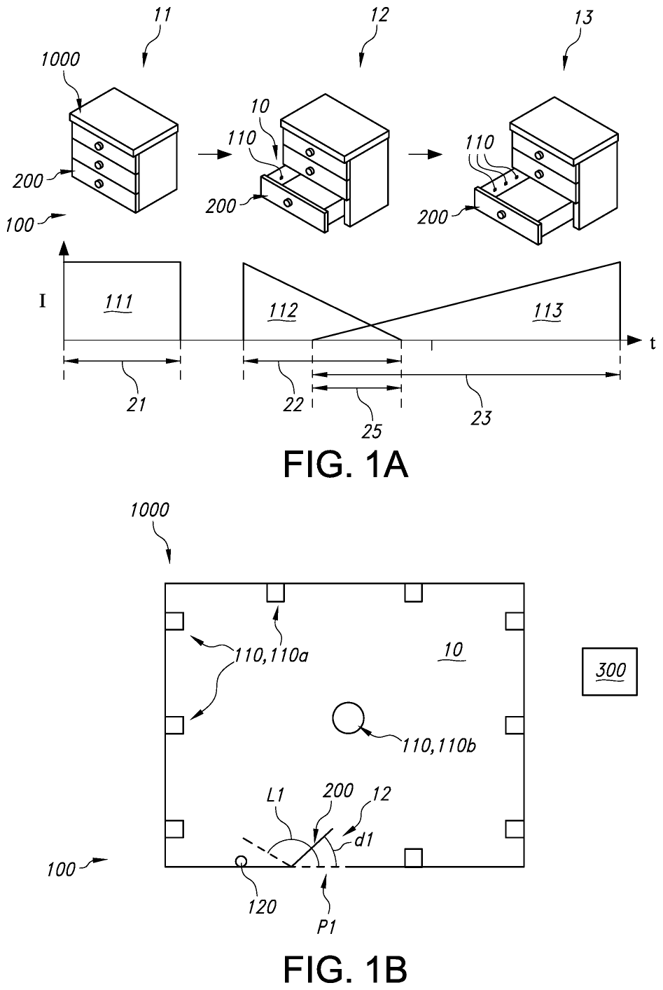

FIG. 1A schematically depicts an embodiment of the system 100 for disinfecting at least part of a closeable space 10. In particular, in the depicted embodiment, the closeable space 10 is the inside of a structure 1000, especially wherein the structure 1000 comprises a cabinet, and wherein the closeable space 10 is the inside of a drawer in the cabinet. The system 100 comprises one or more light sources 110 arranged in the closeable space 10, a sensor 120 and a control system 300 (also see FIG. 1B). In a first operational mode one or more of the one or more light sources 110 may be configured to generate first light source light 111 having one or more wavelengths selected from the range of 100-380 nm. In a second operational mode one or more of the one or more light sources 110 may be configured to generate second light source light 112, especially having a centroid wavelength selected from the range of 400-490 nm. In particular, the first light source light 111 and the second light source light 112 may be provided in (and to) the closeable space 10. The sensor 120 may be configured to detect a state or a change in state of the closeable space 10 and to provide a related sensor signal to the control system 300. In particular, the sensor may be configured to detect whether the closeable space is a closed state 11 or in a (fully) open state 13. In further embodiments, the sensor may further be configured to detect whether the system is in a partially open state 12. In embodiments the control system 300 may be configured to select the first operational mode during a first time period 21 when the closeable space 10 is configured in the closed state 11 and configured to select the second operational mode during a second time period 22 when the closeable space 10 is configured in the open state 13.

Specifically, FIG. 1A schematically depicts the closeable space 10 in a closed state 11, in a partially open state 12, and in an open state 13. In particular, FIG. 1A may schematically depict opening of the closeable space 10 over time t, wherein the first light source light 111 is provided during a first time period 21 while the closeable space 10 is in a closed state 11, wherein the second light source light 112 is provided during a second time period 22 while the system is in an open state 13, especially a partially open state 12, and wherein third light source light 113 is provided during a third time period 23 while the system is in the (fully) open state 13. In particular, FIG. 1A may schematically depict the intensities I (in a.u.) of the first light source light 111, the second light source light 112, and the third light source light 113 over time t.

In embodiments, the system 300 may be configured to select the second operational mode while or after the closeable space 10 changes from the closed state 11 to the open state 13, especially wherein the second time period 22 is selected from the range of 0-5 seconds, such as from the range of 0.1-5 seconds, and wherein the second light source light 112 has a second light intensity.

In the depicted embodiment, the control system 300 is configured to gradually decrease the second light intensity over at least part of the second time period 22, as indicated by the reducing intensity I of the second light source light 112 over time t. In particular, in the depicted embodiment, the control system 300 may be configured to gradually decrease the second light intensity during (all of) the second time period 22.

In further embodiments, the second time period 22 may be selected from the range of 1-5 seconds, and the control system 300 may be configured to gradually decrease the second light intensity over a final part of the second time period 22, wherein the final part is at least 1 second. In such embodiments, the second light intensity may especially be constant during at least part of the second time period 22, such as at during an initial part of the second time period 22, especially wherein the initial part is at least 1 second.

In the depicted embodiment, the system 100, especially the control system 300 may have a third operational mode. In particular, in the third operational mode one or more of the one or more light sources 110 may be configured to generate third light source light 113. The third light source light may especially be white light. In embodiments, the third light source light may have a correlated color temperature selected from the range of 1800-20000 K, especially from the range of 1800-6000 K, such as from the range of 2500-5000 K, especially from the range of 2700-4500 K, such as from the range of 3000-4000 K. The control system 300 may especially be configured to select the third operational mode during a third time period 23 when the closeable space 10 is configured in the open state 13.

In further embodiments, the third light source light may have a color rendering index (CRI) of at least 65, such as at least 70, especially at least 75. In further embodiments, the third light source light may have a color rendering index (CRI) of at least 80, especially at least 85, such as at least 90, especially at least 95.

In further embodiments, the third light source light may have a color rendering index for reference color 9 (red) (also: “R9”) of at least −30, such as at least −10. In further embodiments, the third light source light may have an R9 of at least 0, such as at least 10, especially at least 30, such as at least 50. In further embodiments, the third light source light may have an R9 of at least 60, such as at least 70, especially at least 80.

In the depicted embodiment, the system 100, especially the control system 300, may be configured to gradually change from the second operational mode to the third operational mode, i.e., the control system may after opening of the closeable space 10 gradually decrease the second light intensity and increase the third light intensity over time t. In particular, in the depicted embodiment, the second time period 22 and the third time period 23 temporally overlap during a transitional time period 25, wherein the control system 300 is configured to gradually decrease (the second light intensity of) the second light source light 112 and to gradually increase (the third light intensity of) the third light source light 113 during the transitional time period 25.

In further embodiments, the second operational mode and the third operational mode may be temporally separated, especially wherein the third operational mode is temporally arranged after the second operational mode.

FIG. 1B schematically depicts an embodiment wherein the structure 1000 is a walled space defining a closeable space 10, especially a room.

FIG. 1B schematically depicts a plurality of light sources 110, wherein a first subset of first light sources 110a are configured to provide first light source light 111, and wherein a second light source 110b of the plurality of light sources 110 may be configured to provide the second light source light 110 and optionally the third light source light.

FIGS. 1A and 1B may further schematically depict a structure 1000 comprising the system 100 according to the invention, wherein the structure 1000 defines the closeable space 10. The structure 1000 may comprise a moveable element 200 for opening and closing the closeable space 10, especially wherein the moveable element 200 is selected from the group comprising a lid, a door, and a drawer. In particular, the moveable element 200 may be a drawer in FIG. 1A and a door in FIG. 1B. In embodiments, the sensor 120 may be configured to determine a state or a change in state of the closeable space based on a position of the moveable element 200, and especially to provide a related sensor signal to the control system.

In particular, the sensor 120 may be configured to detect whether the closeable space 10 is in a closed state 11, a partially open state 12 or an open state 13.

In embodiments, the moveable element 200 may be arranged at a first [closed] [end] position P1, such as a drawer being fully shut, or such as a door being arranged in a door frame. In such embodiments, the moveable element 200 may be configured moveable up to a movement limit L1 from the first position P1, such as a length or such as a number of degrees (w.r.t a rotation). In particular, during use the moveable element 200 may have a movement distance d1 (also “distance d1”) to the first position P1.

Hence, in embodiments, the sensor 120 may be configured to determine a parameter related to the movement distance d1 of the moveable element 200 to the first position P1, especially the movement distance d1, and to provide a related sensor signal to the control system.

In further embodiments, the control system 300 may determine the state or a change in state of the closeable space 10 based on the movement distance d1. For example, the closeable space 10 may be (considered to be) in the closed state 11 when d1≤0.03*L1; in the partially open state 12 when 0.03*L1≤ d1≤0.8*L1; and in the open state 13 when 0.8*L1≤d1.

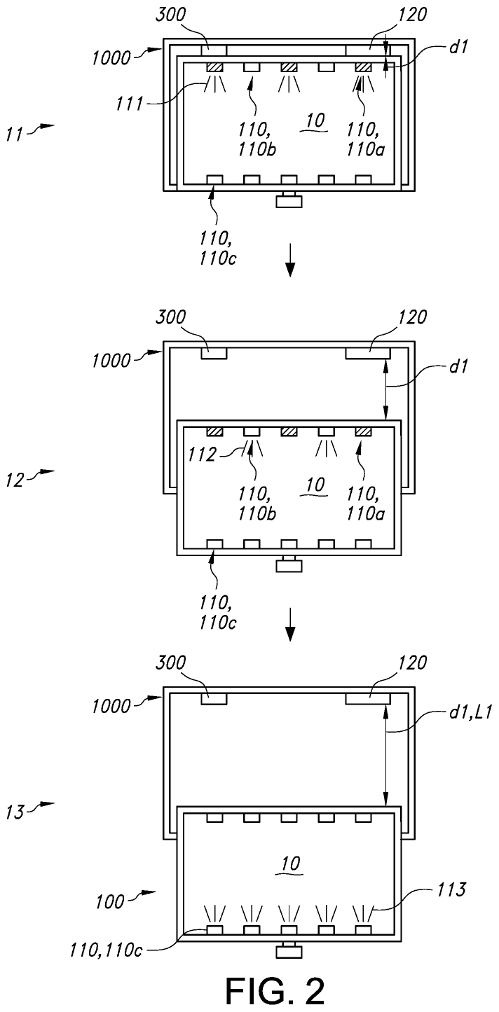

FIG. 2 schematically depicts a further embodiment of the system 100 and the structure 1000 defining the closeable space 10. In particular, the structure may comprise a cabinet and a moveable element 200, especially a drawer. In particular, FIG. 2B may schematically depict a succession of states of the closeable space 10; starting from a closed state 11 to a partially open state 12, to a (fully) open state 13.

In particular, the sensor 120 may be configured to sense a distance d1 between the moveable element 200 and a first position P1 (of the moveable element).

In the closed state 11, the system 100 may be in the first operational mode, wherein one or more first light sources 110, 110a of the one or more light sources 110 may be configured to provide first light source light 111 to the closeable space 10.

As the closeable space 10 is opened and transitions from the closed state 11 to the partially open state 12, the control system may select the second operational mode, wherein one or more second light sources 110, 110b of the one or more light sources 110 may be configured to provide second light source light 112 to the closeable space 10 during a second time period 22.

Once the closeable space is in the open state 13, or after expiration of (at least part of) the second time period 22, the control system 300 may select the second operational mode, wherein one or more third light sources 110, 110c of the one or more light sources 110 may be configured to provide third light source light 112 to the closeable space 10.

In the depicted embodiment, the one or more light sources 110 are divided into the first light sources 110a, the second light sources 110b and the third light sources 110c. However, in further embodiments, there may be overlap between the first light sources 110a and the second light sources 110b, or between the first light sources 110a, and the third light sources 110c, or between the second light sources 110b and the third light sources 110c.

The term “plurality” refers to two or more. Furthermore, the terms “a plurality of” and “a number of” may be used interchangeably.

The terms “substantially” or “essentially” herein, and similar terms, will be understood by the person skilled in the art. The terms “substantially” or “essentially” may also include embodiments with “entirely”, “completely”, “all”, etc. Hence, in embodiments the adjective substantially or essentially may also be removed. Where applicable, the term “substantially” or the term “essentially” may also relate to 90% or higher, such as 95% or higher, especially 99% or higher, even more especially 99.5% or higher, including 100%. Moreover, the terms “about” and “approximately” may also relate to 90% or higher, such as 95% or higher, especially 99% or higher, even more especially 99.5% or higher, including 100%. For numerical values it is to be understood that the terms “substantially”, “essentially”, “about”, and “approximately” may also relate to the range of 90%-110%, such as 95%-105%, especially 99%-101% of the values(s) it refers to.

The term “comprise” also includes embodiments wherein the term “comprises” means “consists of”.

The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices, apparatus, or systems may herein amongst others be described during operation. As will be clear to the person skilled in the art, the invention is not limited to methods of operation, or devices, apparatus, or systems in operation.

The term “further embodiment” and similar terms may refer to an embodiment comprising the features of the previously discussed embodiment, but may also refer to an alternative embodiment.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim.

Use of the verb “to comprise” and its conjugations does not exclude the presence of elements or steps other than those stated in a claim. Unless the context clearly requires otherwise, throughout the description and the claims, the words “comprise”, “comprising”, “include”, “including”, “contain”, “containing” and the like are to be construed in an inclusive sense as opposed to an exclusive or exhaustive sense; that is to say, in the sense of “including, but not limited to”.

The article “a” or “an” preceding an element does not exclude the presence of a plurality of such elements.

The invention may be implemented by means of hardware comprising several distinct elements, and by means of a suitably programmed computer. In a device claim, or an apparatus claim, or a system claim, enumerating several means, several of these means may be embodied by one and the same item of hardware. The mere fact that certain measures are recited in mutually different dependent claims does not indicate that a combination of these measures cannot be used to advantage.

The invention also provides a control system that may control the device, apparatus, or system, or that may execute the herein described method or process. Yet further, the invention also provides a computer program product, when running on a computer which is functionally coupled to or comprised by the device, apparatus, or system, controls one or more controllable elements of such device, apparatus, or system.

The invention further applies to a device, apparatus, or system comprising one or more of the characterizing features described in the description and/or shown in the attached drawings. The invention further pertains to a method or process comprising one or more of the characterizing features described in the description and/or shown in the attached drawings. Moreover, if a method or an embodiment of the method is described being executed in a device, apparatus, or system, it will be understood that the device, apparatus, or system is suitable for or configured for (executing) the method or the embodiment of the method, respectively.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

1. A system for disinfecting at least part of a closeable space, wherein the system comprises one or more light sources, a sensor and a control system, wherein in a first operational mode one or more of the one or more light sources are configured to generate first light source light having one or more wavelengths selected from the range of 100-380 nm in at least part of the closeable space, and wherein in a second operational mode one or more of the one or more light sources are configured to generate second light source light having a centroid wavelength selected from the range of 400-490 nm in at least part of the closeable space and the second light source light having at least 80% of its spectral power in the wavelength range of 100-800 nm in the wavelength range of 400-490 nm, wherein the first light source light and the second light source light are provided in the closeable space, wherein the sensor is configured to detect a state or a change in state of the closeable space and to provide a related sensor signal to the control system, wherein the control system; is configured to select the first operational mode during a first time period when the closeable space is configured in a closed state and configured to select the second operational mode during a second time period when the closeable space is configured in an open state, wherein the system is configured to select the second operational mode while or after changing from the closed state to the open state, wherein the second time period is selected from the range of 0.05-10 seconds.

2. The system according to claim 1, wherein the second time period is selected from the range of 0.1-5 seconds.

3. The system according to claim 1, wherein the second light source light has a second light intensity, wherein the control system is configured to gradually decrease the second light intensity over at least part of the second time period.

4. The system according to claim 3, wherein the control system is configured to gradually decrease the second light intensity over a final part of the second time period, wherein the final part is at least 1 second.

5. The system according to claim 1, wherein the control system is configured to select the second operational mode only if the system has been in the first operational mode for a predetermined consecutive period, wherein the predetermined consecutive period is at least 10 minutes.

6. The system according to claim 1, wherein the sensor is configured to detect whether the closeable space is in the closed state or in the open state, and wherein the control system is configured to switch off the first light source light when the closeable space changes from the closed state to the open state.

7. The system according to claim 1, wherein the first light source light has one or more wavelengths selected from the UV-B range and/or from the UV-C range.

8. The system according to claim 1, wherein in a third operational mode one or more of the one or more light sources are configured to generate third light source light having a correlated color temperature selected from the range of 1800-6000 K, wherein the control system is configured to select the third operational mode during a third time period when the closeable space is configured in the open state.

9. The system according to claim 8, wherein the second time period and the third time period temporally overlap during a transitional time period, wherein the control system is configured to gradually decrease the second light source light and to gradually increase the third light source light during the transitional time period.

10. The system according to claim 1, wherein the sensor is configured to detect whether the closeable space is in a partially open state, wherein the system is configured to select the second operational mode while the system is in the partially open state.

11. The system according to claim 3, wherein the control system is configured to reduce the second light intensity when the closeable space changes from the partially open state to the open state to at most 10% of the second light intensity in the partially open state.

12. The system according to claim 1, wherein the sensor is selected from the group of a heat sensor, a motion sensor, a touch sensor, and a light sensor.

13. A structure comprising the system according to claim 1, wherein the structure defines the closeable space, and wherein the structure comprises a moveable element for opening and closing the closeable space, wherein the moveable element is selected from the group comprising a lid, a door, and a drawer, and wherein the sensor is configured to determine whether the closeable space is in the closed state, or in the open state based on a position of the moveable element.

14. The structure according to claim 13, wherein the moveable element may be arranged at a first position, and wherein the moveable element is configured moveable up to a movement limit L1 from the first position, wherein during use the moveable element has a movement distance d1 to the first position, wherein the closeable space is in the partially open state when 0.03*L1<d1<0.8*L1.

15. A kit of parts, wherein the kit of parts comprises one or more light sources, a control system, and a sensor, and optionally a manual, wherein the control system is configured to select a first operational mode and a second operation mode for the one or more light sources, wherein in the first operational mode one or more of the one or more light sources are configured to generate first light source light having one or more wavelengths selected from the range of 100-380 nm, and wherein in the second operational mode one or more of the one or more light sources are configured to generate second light source light having a centroid wavelength selected from the range of 400-495 nm and the second light source light having at least 80% of its spectral power in the wavelength range of 100-800 nm in the wavelength range of 400-490 nm; and wherein the sensor is configured to detect a state or a change in state of the closeable space and to provide a related sensor signal to the control system, wherein the control system is configured to select the first operational mode during a first time period when the closeable space is configured in a closed state and configured to select the second operational mode during a second time period when the closeable space is configured in an open state, wherein the system is configured to select the second operational mode while or after changing from the closed state to the open state, wherein the second time period is selected from the range of 0.05-10 seconds.
